(19) United States
(12) Patent Application Publication
(10) Pub. No.: US 2007/0104765 A1
(43) Pub. Date: May 10, 2007

(54) Livestock and poultry feed additive composition

(76) Inventors: Gary L. Faltys, West Point, NE (US); Kelly F. Lechtenberg, Oakland, NE (US)

Correspondence Address: LAW OFFICES OF ADAM H. JACOBS, Suite 726, 1904 FARNAM STREET, OMAHA, NE, 68102, US

(21) Appl. No.: 11/595,659

(22) Filed: Nov. 9, 2006

Related U.S. Application Data
(60) Provisional application No. 60/734,817, filed on Nov. 9, 2005.

(51) Int. Cl. A61K 36/752; A61K 36/54; A61K 36/61; A61K 36/53; A61K 36/534; A61K 36/8962

(52) U.S. Cl. 424/442; 424/725; 424/736; 424/739; 424/742; 424/754; 424/745; 424/747; 424/778

(57) ABSTRACT

A livestock and poultry feed additive composition includes at least one essential oil derived from at least one of an herb and a spice and containing thymol and carvacrol as its main ingredients, at least one organic acid derived from at least one of citric, fumaric, fulvic and humic acid and an organic pharmaceutically acceptable carrier. The total amount of the essential oil in the composition is in an amount between twenty percent to forty-five percent (20% to 45%) by weight of the composition. The total amount of the organic acid in the composition is in an amount between fifteen percent to fifty-five percent (15% to 55%) of the composition by weight. Finally, the ratio of carvacrol to thymol in the essential oil is between five to one and nine to one (5:1 and 9:1).

### CROSS-REFERENCE TO RELATED PROVISIONAL PATENT

This application claims priority based on a provisional patent, specifically on the Provisional Patent Application Ser. No. 60/734,817 filed Nov. 9, 2005.

### BACKGROUND OF THE INVENTION

1. Technical Field

The present invention relates to compositions for enhancing palatability of animal feed and promoting growth in livestock and poultry and, more particularly, to a composition including at least one essential oil from an herb or spice, at least one organic acid and an organic carrier, whereby the essential oil is present in an amount between twenty percent to forty-five percent by weight of the composition, and the organic acid is derived from a citric, fumaric, fulvic or humic acid (organic acids) and is present in the composition in an amount between fifteen percent to fifty-five percent of the composition by weight.

2. Description of the Prior Art

Feed designed for use in connection with livestock and poultry generally are concerned with two main elements, the first being that the feed be generally inexpensive and second that it have relatively high amounts of nutritional ingredients in order to promote rapid weight gain in the livestock and poultry to decrease the amount of time needed until the livestock and poultry are ready for processing. One should notice, however, that a high degree of palatability is not listed in the crucial elements of livestock and poultry feed compositions, although it should be clear even to one not skilled in the art that an increase in palatability of the feed composition will likely result in an increase in the amount of feed consumed, and thus, with all other things being equal, will result in a decrease in the amount of time needed until processing of the animal can be undertaken. There have been several different compositions proposed in the prior art which increase the palatability of livestock and poultry feed, but many of these use some combination of flavorings and spices which are intended to replicate the animal's natural feed materials. These prior art compositions have met with some success, but generally do not completely solve the palatability problem. There is therefore a need for an improved palatability-enhancing composition which utilizes improved feed supplements to increase the palatability, and particularly the appetite-enhancing aspects, of the feed.

The ideal feed supplement would also, in addition to enhancing palatability, enhance growth of the livestock and poultry in order to further decrease the amount of time needed until the animal is ready for processing. Although there are several different types of growth-promoting compositions known in the art, very few of these are usable in a feed composition having improved taste, as the taste of the chemical additives generally needs to be disguised via other food additives in order to prevent the animals consuming the feed from noticing them. Ideally, the additive materials would perform the dual function of enhancing the palatability of the feed while simultaneously enhancing the growth of the animal, and in the best of all worlds would include more than one type of food additive so that either or both additives can promote the improved palatability and enhanced growth. There is therefore a need for a feed additive composition which includes at least two palatability-enhancing and growth-promoting additive materials.

Therefore, an object of the present invention is to provide an improved feed additive composition for livestock and poultry.

Another object of the present invention is to provide an improved feed additive composition for livestock and poultry which includes at least one essential oil derived from at least one of an herb and a spice and containing thymol and carvacrol as its main ingredients, at least one organic acid derived from at least one of citric, fumaric, fulvic and humic acid and an organic pharmaceutically acceptable carrier.

Another object of the present invention is to provide an improved feed additive composition for livestock and poultry in which the total amount of essential oil in the composition is between twenty percent to forty-five percent (20% to 45%) by weight of the composition.

Another object of the present invention is to provide an improved feed additive composition for livestock and poultry in which the total amount of organic acid in the composition is an amount between fifteen percent to fifty-five percent (15% and 55%) of the composition by weight.

Another object of the present invention is to provide an improved feed additive composition for livestock and poultry in which the ratio of carvacrol to thymol in the essential oil is between five to one and nine to one (5:1 and 9:1).

Finally, an object of the present invention is to provide an improved feed additive composition for livestock and poultry which is relatively simple to manufacture and which is safe, efficient and effective in use.

### SUMMARY OF THE INVENTION

The present invention provides a livestock and poultry feed additive composition including at least one essential oil derived from at least one of an herb and a spice and containing thymol and carvacrol as its main ingredients, at least one organic acid derived from at least one of citric, fumaric, fulvic and humic acid and an organic pharmaceutically acceptable carrier. The total amount of the essential oil in the composition is in an amount between twenty percent to forty-five percent (20% to 45%) by weight of the composition. The total amount of the organic acid in the composition is in an amount between fifteen percent to fifty-five percent (15% to 55%) of the composition by weight. Finally, the ratio of carvacrol to thymol in the essential oil is between five to one and nine to one (5:1 and 9:1).

The present invention thus provides an improved livestock and poultry feed additive composition which is superior in many respects to those feed additives currently found in the prior art. For example, the specific restrictions of the ratios of essential oils and organic acids have been found to both enhance the palatability of the feed while simultaneously enhancing the growth of the animal. Furthermore, because the present invention utilizes organic acids of several different kinds, adjustment of the amounts and ratios of the specific organic acids used will allow for tailoring of the supplement to various species of livestock. It is thus seen that the present invention provides a substantial improvement over those supplements found in the prior art.

### DESCRIPTION OF THE PREFERRED EMBODIMENT

The present invention is a feed additive composition which includes at least one essential oil derived from an herb or spice such as anise, cinnamon, cloves, eucalyptus, garlic, lemon, oregano, peppermint, rosemary or thyme and an organic acid derived from citric, fumaric, fulvic and/or humic acid. The composition also preferably includes an organic carrier, such as anhydrous dextrose, maltodextran, potassium chloride, salt, organic products, calcium carbonate, inert clay and/or propylene glycol. Each of these elements are mixed together in various ratios depending on the intended use of the feed additive composition, but it is an important feature of the present invention that the essential oil be present in the composition in an amount between twenty percent to forty-five percent (20% to 45%) by weight, and the organic acid be used in the feed additive composition in a percentage of approximately fifteen percent to fifty-five percent (15% to 55%) of the composition by weight, with the remainder of the composition consisting of the organic carrier. It should also be noted that the materials described above from which the essential oils and organic acids are obtained are representative of the preferred types of herbs, spices, and acids to be used as flavoring and growth-promoting compounds, but the present composition may be composed of other types of essential oils and organic acids so long as the essential oils generally improve the palatability of the feed and also promote growth of the livestock or poultry eating the feed having the feed additive composition of the present invention added thereto.

It has been found that the essential compounds of the essential oil which enhance the palatability of the feed additive composition and also promote growth of the livestock and/or poultry are the two compounds carvacrol (2-methyl-5-isopropylphenol) and thymol (5-methyl-2-isopropylphenol), which are derived as part of the production method for making the essential oil from the various herbs and spices. It should be noted that the methods by which carvacrol and thymol are obtained are well-known to the prior art and therefore a further discussion of those methods will not be undertaken in this disclosure. It is preferred that the ratio of carvacrol to thymol in the essential oils will range between five to one to nine to one (5:1 to 9:1) in order to provide the best combination of enhanced palatability and enhanced growth promotion. Alternatively, the essential oil might also contain eucalyptol (1,8-cineole, 1,8-epoxy-P-methane) which also provides enhanced palatability and enhanced growth promotion, although it is to be understood that the primary compounds of the essential oil will be the carvacrol and thymol compounds.

Also, it should be noted that the derivation of the organic acids from the citric, fumaric, fulvic and/or humic acids involve processes which are well known in the prior art and likewise will not be discussed in detail in this disclosure. However, it has been found that the combination of the essential oil and organic acid significantly increase the palatability-enhancing and growth-enhancing aspects of the feed additive composition of the present invention as opposed to those various feed additives described in the prior art which include only essential oils. Also, it has been found that the ratios described previously in which the essential oil is present in the feed additive composition in an amount between twenty percent and forty-five percent (20% to 45%) by weight, the organic acid is used in amounts between fifteen percent and fifty-five (15% to 55%) percent by weight, and the remainder comprises an organic carrier producing a feed additive composition which is significantly improved over those feed additives found in the prior art, particularly those which are intended to provide pharmaceutical compositions for substituting antibiotics and sulphamide-based drugs such as those described in U.S. Pat. Nos. 6,106,838 and 6,322,825. While these pharmaceutical compositions disclose the use of essential oils in ranges in the lower and higher ranges of percentage composition, it has been found that the use of the essential oils in the middle range of composition percentage actually provides the most significant palatability enhancement and growth enhancement, and the inclusion of the organic acids only serves to enhance the performance of the overall feed additive composition of the present invention. It further is an important feature of the present invention that the growth-enhancement aspects of the combination of the essential oils and the organic acids will likely have greater impact on the livestock and poultry than the palatability enhancement aspects, simply because many of the animals to which the present invention will be fed do not have well-developed senses of taste. That being said, however, the appetite-enhancing aspects of the present invention will encourage increased consumption of the feed having the feed additive composition added thereto, which will accomplish the same intended result of increasing the animal growth rate.

It should further be noted that the organic acids and essential oils have a significant impact on the gastrointestinal physiology of the ingesting animals which leads to increased growth rates in the animals. For example, the organic acids and essential oils act to increase enzyme production and further alter the rate of passage of feedstuffs through the gastrointestinal tract. These features of the two main components of the feed additive composition of the present invention function to increase the digestibility of nutrients, and the organic acids also increase mineral absorption.

The organic acids and essential oils also alter the microflora of the gastrointestinal tract thereby altering the digestion of feedstuffs. The essential oils act to decrease the ammonia-producing bacteria in ruminants and thereby decrease the conversion of amino acids to ammonia, which are then available for growth. The combination of the organic acids and essential oils thus contributes greatly to increase the growth rate of the livestock and poultry, a combination not found in the prior art.

The combination of the organic acids and essential oils also has a significant impact on palatability and appetite for many different animals. For example, combining organic acids and essential oils in swine feeds will produce greater feed intake as compared to control diets and control plus essential oils. Also, the essential oils will act as an antioxidant and thus prevent fats from becoming rancid. The antioxidant characteristics as thus described would significantly enhance palatability of essential oils, and therefore the animals will consume more feed.

### EXAMPLES 1-3

Examples 1, 2 and 3 concern the preparation of the water and feed additive composition of the present invention in powder, water-soluble, and syrup form, each of which would be used in different fashions to be added to animal feed.

POWDER FORM

| Ingredient | Young Animal | Adult Animal |
|---|---|---|
| Organic Carrier | 30% | 50% |
| Organic Acids | 50% | 15% |
| Essential Oils | 20% | 35% |

The preparation is blended in a ribbon mixer where the organic carrier is initially added to the mixer. Organic acids and essential oils are added to the organic carrier and the mixture is blended for 5 to 7 minutes. The resulting powder is packaged in 0.45 kg heat-sealed aluminum foil pouches or 10 kg plastic pails. The powder is added to complete feeds for animals or poultry.

WATER SOLUBLE POWDER

| Ingredient | Percentage |
|---|---|
| Citric Acid | 51.75% |
| Anhydrous Dextrose | 5.00% |
| Maltodextran | 2.00% |
| Essential Oils | 41.25% |

The water soluble powder is prepared in a ribbon mixer in an environmentally controlled room. The mixer is charged with citric acid, anhydrous dextrose and maltodextran. The essential oils are added to the above ingredients and blended 5 to 7 minutes. The resulting water soluble powder is packaged in 0.45 kg heat-sealed aluminum foil pouches or 10 kg plastic pails. The water-soluble powder is added to drinking water of animals and poultry via a proportioner.

SYRUP

| Ingredients | Percentage |
|---|---|
| Propylene glycol | 43.75% |
| Citric Acid | 15.00% |
| Essential Oils | 41.25% |

A syrup is prepared by adding propylene glycol (1, 2 propandiole) to a colloidal mixer and heating the liquid to approximately 50° C. Citric acid and essential oils are added and the resulting mixture is blended for 15 minutes. The syrup is allowed to cool and packaged in 0.5 liter plastic bottles. The syrup is added to drinking water of animals and poultry via a proportioner.

It is to be understood that numerous additions, substitutions, and modifications may be made to the feed additive composition of the present invention which fall within the intended broad scope of the above description. For example, the composition, although intended for use with livestock and poultry, may be used with virtually any type of animal in which increased consumption of feed and therefore increased growth is desired. Furthermore, although the essential oil has been described as being derived from various types of herbs and spices, the essential oil may be derived from virtually any type of appropriate foodstuff, so long as at least one of the compounds of carvacrol, thymol, and eucalyptol are included in the feed additive composition of the present invention. Furthermore, the organic acid may be derived from other types of acids known in the prior art, so long as the organic acid which is included in the feed additive composition serves both to enhance palatability and promote the growth component of the feed additive composition. Likewise, the organic carrier used in connection with the present invention may be drawn from various types of inorganic and organic carriers used in feed additive compositions, so long as it is generally inert and provides an appropriate carrier and/or binder for the feed additive composition. Finally, it should be noted that the ratios of elements in the present invention may be modified or changed so long as they stay within the defined limits of the essential oil in an amount between twenty percent to forty-five percent (20% to 45%), the organic acid in a concentration of fifteen percent to fifty-five percent (15% to 55%) by weight, and the organic carrier comprising the remainder of the feed additive composition.

There has therefore been shown and described a feed additive composition for enhancing palatability and enhancing animal growth which accomplishes at least all of its intended purposes.

I claim:

1. A livestock and poultry feed additive composition comprising:
at least one essential oil derived from at least one of an herb and a spice and containing thymol and carvacrol as its main ingredients;
at least one organic acid derived from at least one of citric, fumaric, fulvic and humic acid;
an organic pharmaceutically acceptable carrier wherein;
the total amount of said essential oil in said composition being present in an amount between twenty percent to forty-five percent (20% and 45%) by weight of said composition;
the total amount of said organic acid in said composition being present in an amount between fifteen percent to fifty-five percent (15% and 55%) of said composition by weight; and
the ratio of carvacrol to thymol in said essential oil is between five to one and nine to one (5:1 and 9:1).

2. The livestock and poultry feed additive composition of claim 1 wherein said at least one essential oil is derived from an herb or spice selected from the group consisting of anise, cinnamon, cloves, eucalyptus, garlic, lemon, oregano, peppermint, rosemary and thyme.

3. The livestock and poultry feed additive composition of claim 1 wherein said at least one organic acid is derived from and acid selected from the group consisting of citric, fumaric, fulvic and humic acid.

4. The livestock and poultry feed additive composition of claim 1 wherein said organic pharmaceutically acceptable carrier is selected from the group consisting of anhydrous dextrose, maltodextran, potassium chloride, salt, calcium carbonate, inert clay and propylene glycol.

5. A livestock and poultry feed additive composition comprising:
at least one essential oil derived from at least one of an herb and a spice selected from the group consisting of anise, cinnamon, cloves, eucalyptus, garlic, lemon, oregano, peppermint, rosemary and thyme and containing thymol and carvacrol as its main ingredients;
at least one organic acid derived from at least one of citric, fumaric, fulvic and humic acid;
an organic pharmaceutically acceptable carrier selected from the group consisting of anhydrous dextrose, maltodextran, potassium chloride, salt, calcium carbonate, inert clay and propylene glycol, wherein;
the total amount of said essential oil in said composition being present in an amount between twenty percent to forty-five percent (20% and 45%) by weight of said composition;
the total amount of said organic acid in said composition being present in an amount between fifteen percent to fifty-five percent (15% and 55%) of said composition by weight; and
the ratio of carvacrol to thymol in said essential oil is between five to one and nine to one (5:1 and 9:1).
